(19) United States
(12) Patent Application Publication
(10) Pub. No.: US 2016/0296357 A1
(43) Pub. Date: Oct. 13, 2016

(54) Removable and Adjustable Cast

(71) Applicants: Kenneth C. Westover, Fort Worth, TX (US); Jonathan T. Suder, Fort Worth, TX (US); Brett M. Pinkus, Dallas, TX (US)

(72) Inventors: Kenneth C. Westover, Fort Worth, TX (US); Jonathan T. Suder, Fort Worth, TX (US); Brett M. Pinkus, Dallas, TX (US)

(21) Appl. No.: 15/095,414

(22) Filed: Apr. 11, 2016

Related U.S. Application Data
(60) Provisional application No. 62/145,664, filed on Apr. 10, 2015.

(51) Int. Cl. A61F 5/01; A61F 5/058

(52) U.S. Cl. CPC A61F 5/012; A61F 5/0104; A61F 5/05841; A61F 5/05858

(57) ABSTRACT

An orthopedic cast is provided that is removable and/or adjustable to allow for examination of the injury, cleaning, treatment, and adjustment of the tightness of the fit. The cast may include one or more longitudinally extending openings along at least a portion of the length of the cast. The cast may further include at least one adjustment device that is adjustable to increase or decrease the tightness of the fit of the cast, at least one locking device that releasably couples the cast together at one or more longitudinal openings, and/or at least one joining device that joins together one or more longitudinally extending openings of the cast.

### PRIORITY

This application claims the benefit of priority of U.S. Provisional Patent Application No. 62/145,664, filed on Apr. 10, 2015.

### FIELD OF THE INVENTION

The present invention relates generally to orthopedic casts, and more specifically, to removable and adjustable orthopedic casts.

### BACKGROUND

One of the most common orthopedic problems is broken bones. Orthopedic surgeons cast broken limbs, extremities, or other anatomical structures to ensure their proper recovery. In one common casting technique, a cotton sleeve, liner, or stockinette is slid over the injured area and a cotton gauze may be wrapped over the sleeve, liner, or stockinette to provide padding and a certain degree of rigidity. Subsequently, a casting material made of plaster or fiberglass is applied as the final layer of the cast to form a hard shell.

A patient wearing an orthopedic cast may have a need for the cast to be removed for any of a number of reasons, including but not limited to, examination of the injury, cleaning, treatment a rash or infection, or to allow the patient to bathe without having to keep the cast dry. The patient may also initially undergo swelling at the area around the broken bone that later subsides and which may present a need to increase or decrease the tightness of the fit of the cast over time.

Accordingly, there is a need for a removable and adjustable orthopedic cast to address the above problems.

### SUMMARY

According to one embodiment, an orthopedic cast comprises a padding layer and a shell comprised of a hard material disposed over the padding layer. The shell may include at least one longitudinal opening extending along at least a portion of the length of the cast. At least one adjustment device may be provided that secures the cast around an anatomical structure of a patient's body and is adjustable to increase or decrease the tightness of the fit of the cast around the anatomical structure. The adjustment device may include at least one locking device that is lockable to preclude the adjustment device from being adjusted to increase or decrease the tightness of the fit of the cast.

According to one embodiment, the orthopedic cast may further comprise at least one locking device that releasably couples the cast together at the at least one longitudinal opening.

According to one embodiment, the orthopedic cast may include two or more longitudinal openings extending along at least a portion of the length of the cast. The orthopedic cast may further comprise at least one joining device securing together the cast at least one of the at least two longitudinal openings.

According to one embodiment, the adjustment device may comprise one or more bands that wrap around the circumference of the cast to secure the cast around an anatomical structure of a patient's body. The bands may comprise one or more wires that are adjustable to increase or decrease the tightness of the bands.

According to one embodiment, the adjustment device may comprise an insert disposed on a strip of material around which the cast is fabricated.

According to one embodiment, the adjustment device may comprise at least one insertable or inflatable section disposed between the shell of the cast and the anatomical structure of the patient.

According to one embodiment, the cast may include an adjustable section at the location of a joint.

According to one embodiment, the cast may include additional items or features that are built into, attached to, mounted on, or otherwise disposed on the cast. The cast may include a window formed in the cast in which an additional item can be inserted.

These and other features of this invention are described in, or are apparent from, the following detailed description of various exemplary embodiments of this invention.

### BRIEF DESCRIPTION OF THE DRAWINGS

Exemplary embodiments of this invention will be described with reference to the accompanying figures.

FIG. 1 depicts a conventional orthopedic cast on a patient's arm.

FIG. 2 depicts an embodiment of a cast of the present invention in a closed and locked configuration.

FIG. 3 depicts the embodiment of the cast in FIG. 2 in an unlocked and open configuration.

FIG. 4 depicts the inside of the embodiment of the cast in FIG. 2.

FIG. 5 depicts an embodiment of a joining device joining adjacent longitudinal sides of two components of a cast.

FIG. 6 depicts an embodiment of a locking device fixedly securing together adjacent longitudinal sides of two components of a cast.

FIG. 7 depicts another embodiment of a locking device in the form of an insert around which the cast may be fabricated.

FIG. 8 depicts an embodiment of a cast formed with one longitudinal opening cut along the cast.

FIG. 9 depicts an embodiment of an adjustment device in the form of a band for increasing or decreasing the tightness of the fit of the cast around the anatomical structure.

FIG. 10 depicts another an embodiment of an adjustment device in the form of a band for increasing or decreasing the tightness of the fit of the cast around the anatomical structure.

FIG. 11 depicts another embodiment of an adjustment device in the form of an insertable section for increasing or decreasing the tightness of the fit of the cast around the anatomical structure.

FIG. 12 depicts an embodiment of a cast including an adjustable section at a joint.

### DETAILED DESCRIPTION OF THE EXEMPLARY EMBODIMENTS

In the following detailed description of the exemplary embodiments, reference is made to the accompanying drawings, which form a part hereof, and in which are shown by way of illustration example embodiments in which the invention may be practiced. It is to be understood that other embodiments may be utilized and structural changes may be made without departing from the scope of the present invention as defined by the appended claims. The embodiments shown herein are intended to be illustrative of various aspects and benefits of the invention and are not intended to limit the scope of the invention to the embodiments shown.

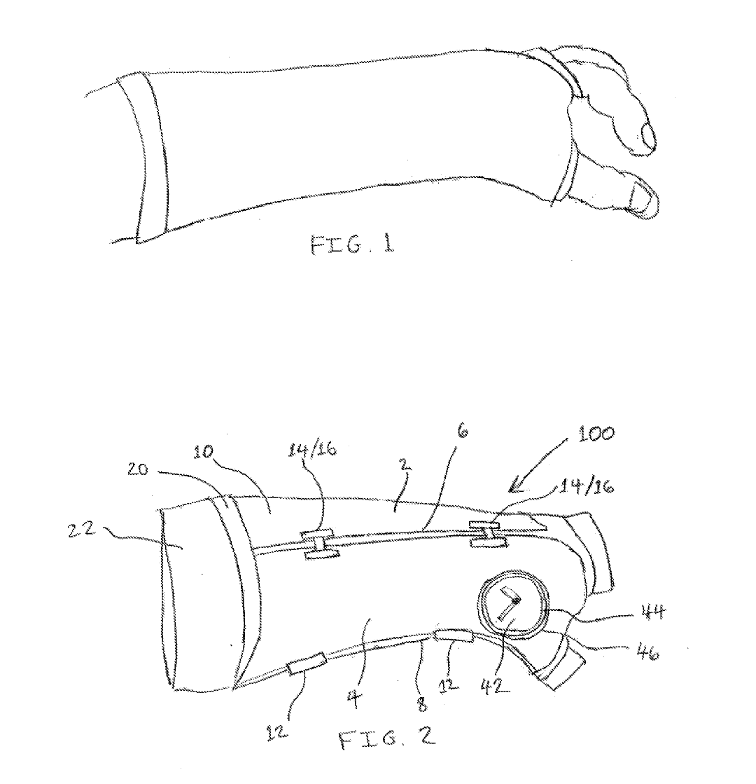

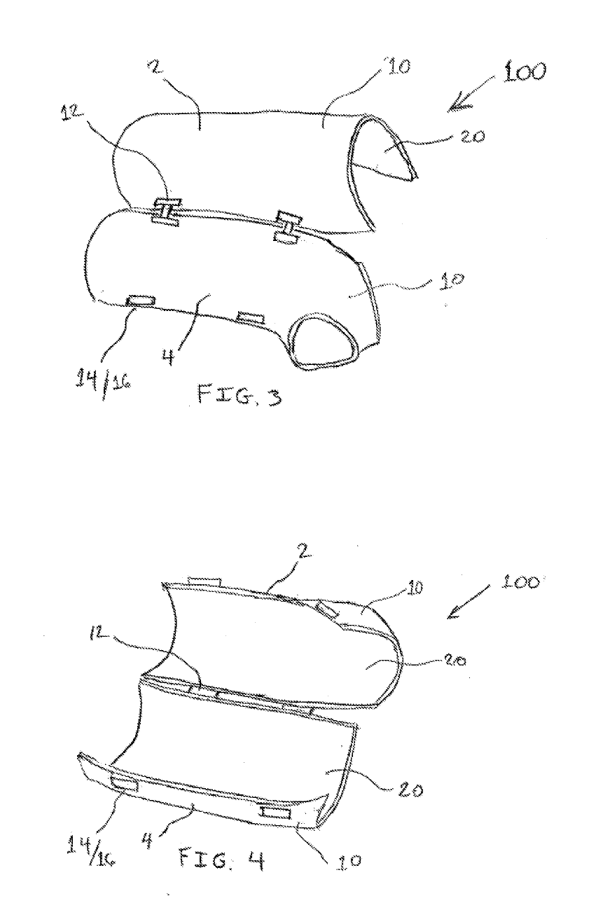

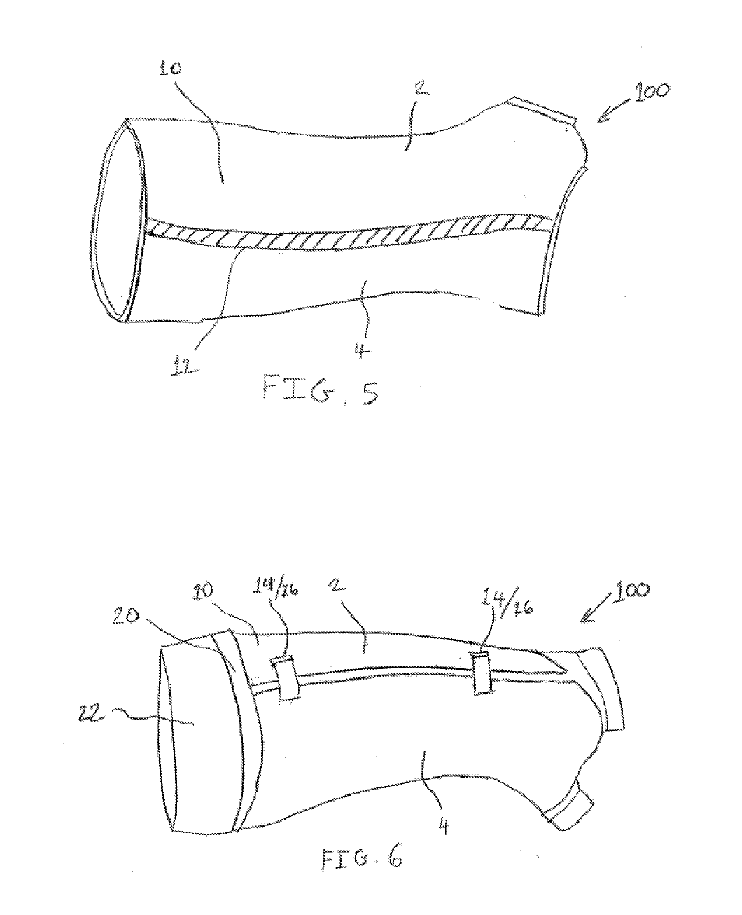

FIGS. 2-4 depict an example embodiment of a removable and adjustable orthopedic cast 100. The cast 100 includes an outer shell 10 to stabilize and hold anatomical structures in place until healing is confirmed, most often broken bones and/or damaged ligaments in a patient's arm, leg, neck, spine, or the like. The outer shell 10 may be made of a variety of different hard or hardenable materials to provide stability. For example, the two most typical kinds of casting materials include plaster and fiberglass, but other casting materials may be used such as a thermoplastic.

The cast 100 may include a padding layer 20 disposed between the cast and the patient's skin to make it soft and to provide padding around bony areas, such as the wrist, ankle, knee or elbow. The padding layer 20 may be formed as a lining on the inside of the cast and/or may be separate padding 22 placed between the cast and the patient's skin, such as a sleeve, liner, or stockinette. For example, the padding layer 20 may be comprised of cotton, felt, foam, or other soft padding materials. The padding layer 20 may be made of a material that can stiffen after it is set in place to form a mold for the removable cast. The padding layer 20 may be made a waterproof material that would allow the cast to get wet. In addition or in the alternative, the cast may include a separate waterproof liner disposed on the inside or outside of the cast.

In one embodiment such as shown in FIG. 2, the cast 100 may be comprised of two or more longitudinal components 2, 4 spanning the length or a portion of a length of the cast. The components 2, 4 may be separately fabricated pieces or may be formed by cutting longitudinal incisions or kerfs in the cast 100. The components 2, 4 are then joined or fit together and placed around an anatomical structure of a patient. In another embodiment such as shown in FIG. 8, the cast 100 may be one monolithic component that includes an elongated incision, kerf, or opening that extends longitudinally along the length or a portion of the length of the cast to form an opening through which an anatomical structure of a patient can be accessed and can be placed into or removed the cast. The cast may be pre-manufactured or may be molded by a medical practitioner.

The two or more components 2, 4 of the cast 100 may be joined together at one or both longitudinal sides 6, 8 by one or more joining device 12. For example, the joining device 12 may be a joint, hinge, fastener, lock, latch, buckle, clasp, pin, magnet, binding, belt, tie, cord, strap, wire or the like. Further, for example, as shown in FIG. 5, the joining device may be a hinge formed of silicon or another adhesive material between the adjoining longitudinal sides 6, 8 of the two components 2, 4. When two components 2, 4 are joined along one adjacent longitudinal side 6, the two joined components may be separated along the other longitudinal side 8 while still remaining connected. The components 2, 4 may be joined during or after the making of the cast. For example, the joining device 12 may be an insert disposed on a strip, sleeve, or template around which the cast is formed or which may be secured to the cast during or after assembly. The strip, sleeve, or template may provide guidance to the medical practitioner as to where and/or how deep to cut longitudinal incisions or kerfs into the cast.

The cast 100 may include one or more locking device 14 that releasably secures the two or more longitudinal components 2, 4 of the cast together. The locking device 14 can be unlocked so that the two or more components 2, 4 of the cast can be separated to allow the patient's anatomical structure for adjustment or removal from the cast. For example, it may be desirable to remove the cast for examination of the injury, cleaning, or allowing the patient to bathe without having to keep the cast dry. The cast can then be replaced around the patient's anatomical structure and the locking device 14 can then be locked to fixedly secure the cast in place. The locking device 14 may allow the patient to unlock the device at any time or may be limited to be unlocked in only certain situations, such as by a medical practitioner, a caretaker, or in the case of a child, the patient's parents.

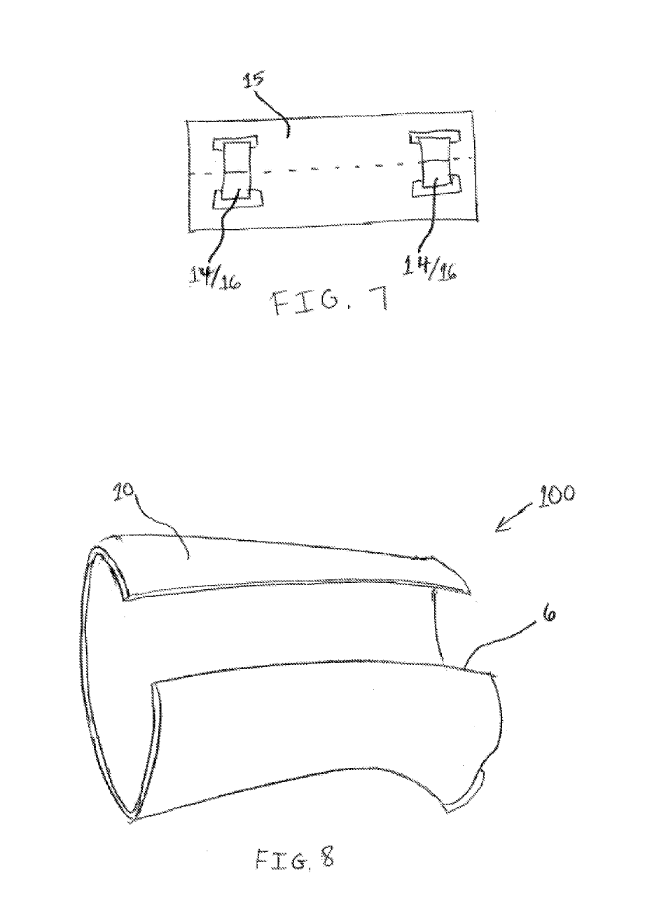

For example, the locking device 14 may comprise a fastener, lock, latch, buckles, clasp, pin, magnet, binding, belt, tie, cord, band, strap, wire or the like. The locking device 14 may be engaged to lock mechanically, magnetically, electronically, or the like. Unlocking of the locking device 14 may require a wrench, driver, mechanical key, magnetic key, electronic key or key fob, RFID key or key fob, or the like. The locking device 14 may be located at or near one adjacent longitudinal side of two components 2, 4 or it may wrap around the circumference or a portion of the circumference of the cast. Locking devices 14 may be placed at one or more locations along the length or a portion of the length of the cast. The locking device 14 may be attached to the cast during or after the fabrication of the cast. For example, as shown in FIG. 7, the locking device 14 may be an insert comprising the locking device disposed on a strip, sleeve, or template 15 around which the cast is formed or which may be secured to the cast during or after assembly. The strip, sleeve, or template 15 may provide guidance to the medical practitioner as to where and/or how deep to cut longitudinal incisions or kerfs into the cast.

The cast 100 may include one or more adjustment device 16 for adjusting the tightness and fit of the cast. The tightness may be adjustable at one or more locations along the length of the cast to provide a more custom fit or to adjust for the occurrence or subsidence of swelling. For example, when using two or more adjustment devices 16, each device 16 may be adjusted independently. The adjustment device 16 may be an insert disposed on a strip, sleeve, or template around which the cast is formed or it may be secured to or disposed on the cast during or after assembly. The strip, sleeve, or template may provide guidance to the medical practitioner as to where and/or how deep to cut longitudinal incisions or kerfs into the cast.

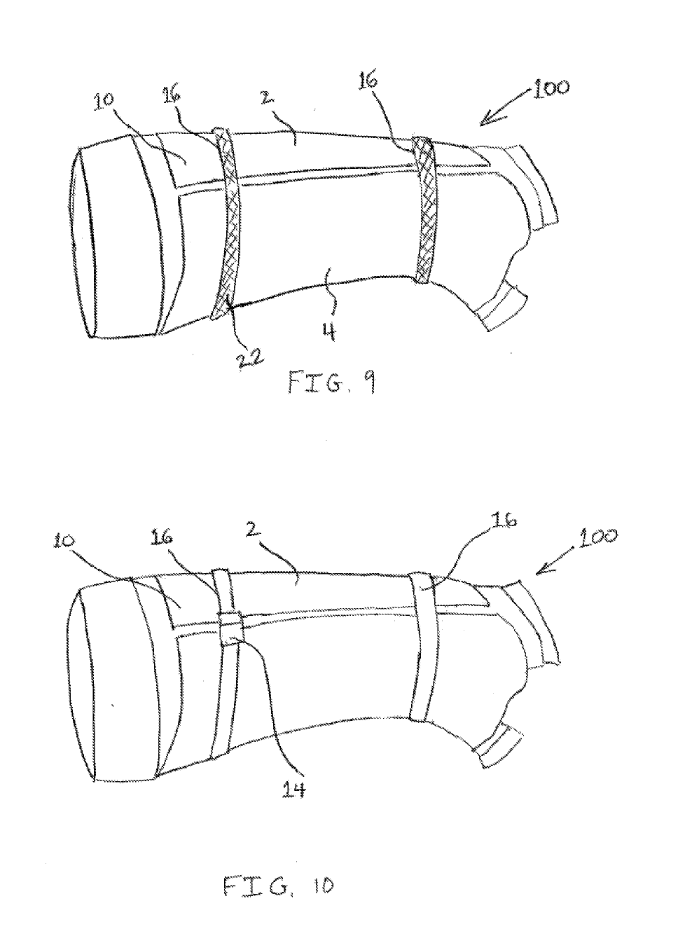

In one embodiment, an adjustment device 16 may include one or more components, such as fastener, latch, buckle, clasp, pin, magnet, binding, belt, tie, cords, bands, strap, wire or the like, or any combination thereof. The adjustment device 16 may include a locking device 14 such as shown in FIG. 10. For example, the locking device 14 may be adjustable to increase or decrease the tightness of the fit, such as by changing the setting in a clasp or ratchet or by turning a knob, screw, or other fastener. In another example, the locking device 14 may be unlocked or loosened to allow the one or more other components of the adjustment device 16 to be adjusted to increase or decrease the tightness and locked to preclude adjustment of the adjustment device 16. In another example, one or more components of the adjustment device 16, such as a binding, tie, cord, band, strap, wire or the like wrapping around the circumference or a portion of the circumference of the cast, may be removed and exchanged with tighter or looser fitting component.

In another embodiment such as shown in FIG. 9, at least one component of the adjustment device 16 may comprise one or more wires or ties, cords, bands, straps or the like including one or more wires formed therein or mounted thereto, which can be adjusted to tighten or loosen the adjustment device. For example, the wires 32 may be comprised of a polymer, metal alloy, or the like. The wires 32 may exhibit elasticity, superelasticity, shape memory, or other properties which allow the tightness to be adjusted to tighten or loosen the fit. Shape memory metal alloys can provide for variations in tightness caused by superelastic behavior during a change of the mechanical influences on the material or by shape memory effect during a temperature change above the transformation temperature of the material.

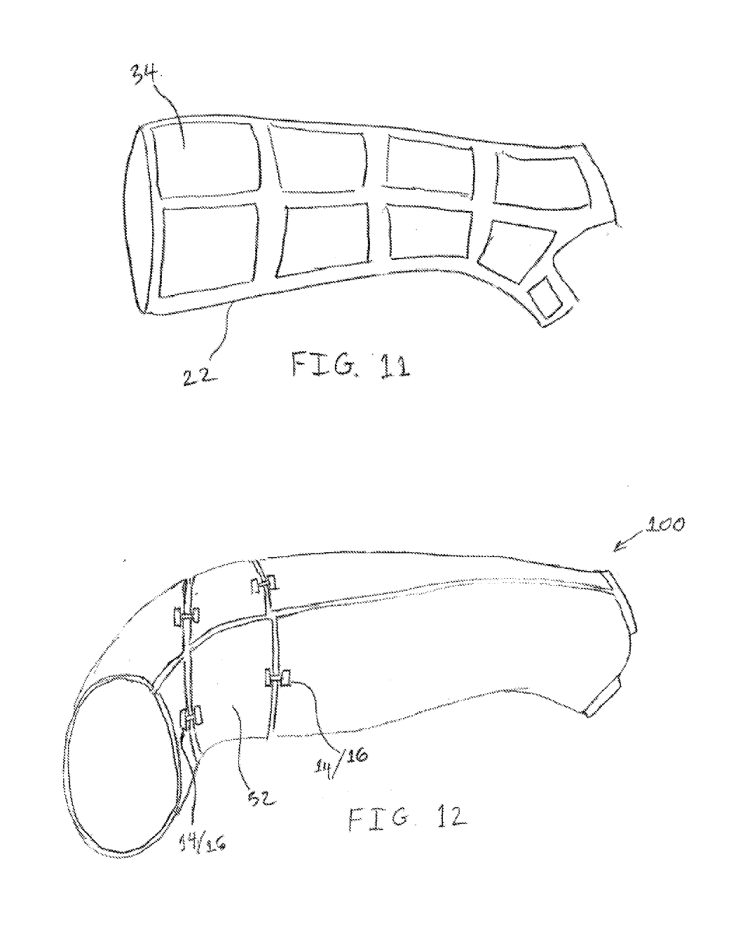

In another embodiment such as shown in FIG. 11, the thickness of the cast 100 may be adjustable at one or more sections. For example, one or more insertable sections 34 may be disposed inside the cast to adjust the tightness and fit at one or more locations. The insertable sections 34 may be comprised of a soft padding material, such as cotton or foam, or of a denser or more rigid material. In another embodiment, the cast may instead include one or more inflatable sections 32 to adjust the tightness and fit. For example, the outer shell 10 of the cast, a layer of the cast 20 or a liner, a sleeve, or a stockinette 22 may include one or more inserts or inflatable sections 34 attached thereto. The insertable or inflatable sections 34 may be removable and replaceable or may be fixedly attached.

The cast 100 may further be adjustable to allow a patient's anatomic features to be set in customized orientations. For example, as shown in an embodiment in FIG. 12, the cast may include an adjustable section 52 at the location of a joint, such as a patient's wrist, elbow, knee, or ankle. In this way, if this cast is pre-manufactured, it may customized to fit different patients and treat various types of injuries

The cast 100 may include other features that are built into, attached to, mounted on, or otherwise disposed on the cast. For example, as shown in the embodiment of FIG. 2, the cast may include an ornamental or other item 42, such as a watch, attached to the outside surface of the cast. The cast may include a window 44 to allow the item 42 to be placed on the inside surface or inside the cast itself such that the item 42 is viewable from the outside surface of the cast. The widow 44 may be formed by cutting out a portion of the cast after fabrication or may be formed by molding the cast around a window insert 46 during fabrication.

Now that exemplary embodiments of the present invention have been shown and described in detail, various modifications and improvements thereon will become readily apparent to those skilled in the art. Accordingly, the spirit and scope of the present invention is to be construed broadly and limited only by the appended claims, and not by the foregoing specification.

What is claimed is:

1. An orthopedic cast, comprising:
a padding layer;
a shell comprised of a hard material disposed over the padding layer, the shell including at least one longitudinal opening extending along at least a portion of the length of the cast;
at least one adjustment device that secures the cast around an anatomical structure of a patient's body and is adjustable to increase or decrease the tightness of the fit of the cast around the anatomical structure.

2. The orthopedic cast of claim 1, wherein the adjustment device includes at least one locking device that is lockable to preclude the adjustment device from being adjusted to increase or decrease the tightness of the fit of the cast.

3. The orthopedic cast of claim 1, further comprising at least one locking device that releasably couples the cast together at the at least one longitudinal opening.

4. The orthopedic cast of claim 1, wherein the shell includes two or more longitudinal openings extending along at least a portion of the length of the cast, further comprising at least one joining device securing together at least one of the two or more longitudinal openings.

5. The orthopedic cast of claim 1, wherein the adjustment device comprises one or more bands that wraps around the circumference of the cast to secure the cast around an anatomical structure of a patient's body.

6. The orthopedic cast of claim 5, wherein the bands comprises one or more wires that are adjustable to increase or decrease the tightness of the adjustment device.

7. The orthopedic cast of claim 1, wherein the adjustment device comprises an insert disposed on a strip of material around which the cast is fabricated.

8. The orthopedic cast of claim 1, wherein the adjustment device comprises an insertable section disposed between the shell of the cast and the anatomical structure of the patient.

9. The orthopedic cast of claim 1, wherein the adjustment device comprises an inflatable section disposed between the shell of the cast and the anatomical structure of the patient.

10. An adjustment device for an orthopedic cast, comprising:
a band that wraps around the circumference of the cast to secure the cast around an anatomical structure of a patient's body;
wherein the band is adjustable to increase or decrease the tightness of the fit of the cast around the anatomical structure.

11. The adjustment device of claim 10, wherein the band comprises one or more wires that are adjustable to increase or decrease the tightness of the adjustment device.
